(19) United States
(12) Patent Application Publication
(10) Pub. No.: US 2025/0114533 A1
(43) Pub. Date: Apr. 10, 2025

(54) ADAPTIVE SYRINGE PLUNGER

(71) Applicant: Hill-Rom Services, Inc., Batesville, IN (US)

(72) Inventors: Taylor Vohland, Batesville, IN (US); Olivia Gubitose Boyce, Cincinnati, OH (US)

(21) Appl. No.: 18/897,654

(22) Filed: Sep. 26, 2024

Related U.S. Application Data
(60) Provisional application No. 63/543,134, filed on Oct. 9, 2023.

(51) Int. Cl. A61M 5/315

(52) U.S. Cl. CPC A61M 5/31513

(57) ABSTRACT

An adaptive syringe plunger assembly includes a syringe and a plurality of syringe interface devices that are each removably couplable with the syringe. Each of the plurality of syringe interface devices increases a surface area for a syringe user to apply force to the syringe to administer the syringe.

### CROSS-REFERENCE TO RELATED APPLICATIONS

This application claims priority to U.S. Provisional Application No. 63/543,134 filed on Oct. 9, 2023, the entire disclosure of which is incorporated herein by reference.

### BACKGROUND

The present disclosure relates to syringes, and particularly, to adaptive syringe plungers. More particularly, the present disclosure relates to syringe interface devices that are configured to be removably coupled with a syringe plunger.

Generally, syringe plungers have small, circular ends. Syringe users, such as clinical staff and individuals using syringes in at-home care, apply force to the small, circular end of the syringe plunger using their thumb. Clinical staff and individuals with lower dexterity or hindered movement of their thumb may have difficulties administering medication from such syringes. Thus, syringe plungers having a larger surface area to which to apply force would be appreciated by syringe users.

### SUMMARY

An apparatus, system, or method may comprise one or more of the features recited in the appended claims and/or the following features which, alone or in any combination, may comprise patentable subject matter:

According to the present disclosure, an adaptive syringe plunger assembly may be provided. The adaptive syringe plunger assembly may include a syringe and a plurality of syringe interface devices. The syringe may be configured to eject fluid. The syringe may include a syringe body and a plunger received, at least partially, inside of the syringe body for slidable movement along a plunger axis. The plunger may have a first end arranged inside of the syringe body and a second end opposite the first end and arranged outside of the syringe body. The plurality of syringe interface devices may be each removably couplable with the second end of the plunger. The plurality of syringe interface devices may be configured to receive force applied by a syringe user to slide the plunger within the syringe body and eject the fluid from the syringe body. Each of the plurality of syringe interface devices may increase a surface area for the syringe user to apply the force to a corresponding one of the plurality of syringe interface devices that is coupled with the second end of the plunge. Each of the plurality of syringe interface devices may have a different shape.

Optionally, at least one of the plurality of syringe interface devices may include a body and an attachment stem formed to include a hole to receive the second end of the plunger therein. The body may include a top wall and a bottom wall opposite the top wall. The attachment stem may extend downwardly away from the bottom wall of the body and may be aligned with the plunger axis. The body of the at least one of the plurality of syringe interface devices may be triangular in shape having three rounded points circumferentially spaced apart from one another relative to the plunger axis. The top wall of the body may curve downwardly toward the attachment stem at each of the three rounded points such that an apex of the body may be aligned with the plunger axis.

Further optionally, the body of the at least one of the plurality of syringe interface devices may be conical in shape. An apex of the body may be aligned with the plunger axis and the top wall may extend downwardly away from the apex.

If desired, the body of the at least one of the plurality of syringe interface devices may be cylindrical in shape. The top wall of the body may be substantially flat. The body may include a side wall extending between and interconnecting the top wall and the bottom wall of the body. The side wall may curve radially inwardly toward the plunger axis to form a notch sized to receive fingers of the syringe user therein.

Alternatively, the bottom wall may have a first end and a second end opposite the first end. The top wall may extend upwardly away from and may be coupled with the first end and the second end of the bottom wall to form a finger-receiving region between the top wall and the bottom wall.

Additionally, the body may include a side wall extending between and interconnecting the top wall and the bottom wall of the body. At least a portion of the side wall may be formed to include finger-receiving grooves sized to receive fingers of the syringe user therein.

Optionally, the hole of the attachment stem of the at least one of the plurality of syringe interface devices may be formed to include threads that mate with the second end of the plunger to removably couple the at least one of the plurality of syringe interface devices with the plunger.

Further optionally, at least a portion of the attachment stem may be formed of an elastomer material. The elastomer material may be configured to deform to removably couple the at least one of the plurality of syringe interface devices with the plunger.

Alternatively, the attachment stem may be formed to include a first attachment portion and a second attachment portion. The first attachment portion may have a tab extending radially away from the first attachment portion and into a tab receiver formed in the second attachment portion to couple the first attachment portion and the second attachment portion together. The first attachment portion and the second attachment portion of the attachment stem may cooperate to form the hole that receives the plunger therein. The first attachment portion and the second attachment portion may be removably coupled together to removably couple the at least one of the plurality of syringe interface devices with the plunger.

Additionally, at least one of the plurality of syringe interface devices may include a hook-and-loop fastener strap formed to include a slit extending therethrough and sized to receive the second end of the plunger therein to removably couple the at least one of the plurality of syringe interface devices with the plunger.

If desired, at least one of the plurality of syringe interface devices may include a body and an attachment stem formed to receive the second end of the plunger therein. The body may include a top wall and a bottom wall opposite the top wall. The attachment stem may extend downwardly away from the bottom wall of the body and may be aligned with the plunger axis. The bottom wall may have a first end and a second end opposite the first end. The top wall may extend upwardly away from and may be coupled with the first end and the second end of the bottom wall to form a finger-receiving region between the top wall and the bottom wall. The attachment stem may include a first attachment arm extending downwardly from the body and a second attachment arm radially spaced apart from the first attachment arm and extending downwardly from the body. Each of the first attachment arm and the second attachment arm may be formed to include a tab extending radially inwardly from a bottom edge of each of the first attachment arm and the second attachment arm. Each of the tabs may be configured to snap under the second end of the plunger to removably couple the at least one of the plurality of syringe interface devices with the plunger.

Optionally, at least one of the plurality of syringe interface devices may include a body, a first attachment arm extending downwardly from the body, and a second attachment arm radially spaced apart from the first attachment arm and extending downwardly from the body. A tensioning unit may extend around the first attachment arm, the second attachment arm, and the plunger to removably couple the plunger with the at least one of the plurality of syringe interface devices.

Further optionally, at least one of the plurality of syringe interface devices may include a body, a first attachment arm extending downwardly from the body, and a second attachment arm radially spaced apart from the first attachment arm and extending downwardly from the body. The first attachment arm and the second attachment arm may be configured to wrap around the plunger to removably couple the at least one of the plurality of syringe interface devices with the plunger.

If desired, the plunger may include a plunger shaft extending between and interconnecting the first end and the second end of the plunger. The plunger shaft may have a first diameter. The second end of the plunger may be a thumb pad. The thumb pad may have a second diameter. The second diameter may be greater than the first diameter. At least one of the plurality of syringe interface devices may include a hollow body and an attachment stem extending downwardly from the hollow body. The attachment stem may have a third diameter that is less than the second diameter of the thumb pad. The attachment stem may be configured to deform so that the thumb pad passes through the attachment stem and is received in the hollow body.

Further according to the present disclosure, an adaptive syringe plunger assembly may be provided. The adaptive syringe plunger assembly may include a syringe and a syringe interface device. The syringe may be configured to eject fluid. The syringe may include a syringe body and a plunger received, at least partially, inside of the syringe body for slidable movement along a plunger axis. The plunger may have a first end arranged inside of the syringe body and a second end opposite the first end and arranged outside of the syringe body. The syringe interface device may be configured to be removably coupled with the second end of the plunger to receive force applied by a palm of a syringe user to slide the plunger within the syringe body and eject the fluid from the syringe body. The syringe interface device may include a body and an attachment stem extending downwardly away from the body to be removably coupled with the second end of the plunger. The syringe interface device may be configured to increase a surface area for the syringe user to apply the force to the syringe interface device.

Optionally, the attachment stem may be formed to include a hole sized to receive the plunger therein. The body may be formed to include a top wall and a bottom wall opposite the top wall. The attachment stem may extend downwardly from the bottom wall of the body and may be aligned with the plunger axis. The body may be triangular in shape having three circumferentially spaced apart points relative to the plunger axis. The top wall of the body may curve downwardly at each of the three points such that an apex of the body may be aligned with the plunger axis.

Further optionally, the body may be conical in shape. An apex of the body may be aligned with the plunger axis. The top wall may extend downwardly from the apex.

If desired, the body may be cylindrical in shape and the top wall of the body may be substantially flat. The body may include a side wall extending between and interconnecting the top wall and the bottom wall of the body. The side wall may curve radially inwardly toward the plunger axis to form a notch sized to receive fingers of the syringe user therein.

Alternatively, the bottom wall may have a first end and a second end opposite the first end. The top wall may extend upwardly away from the first end and the second end of the bottom wall to form a finger-receiving region therebetween the top wall and the bottom wall.

Additionally, the body may include a side wall extending between and interconnecting the top wall and the bottom wall of the body. The side wall may be formed to include finger-receiving grooves sized to receive fingers of the syringe user therein.

Optionally, the hole of the attachment stem may be formed to include threads that mate with the second end of the plunger to removably couple the syringe interface device with the plunger.

Further optionally, at least a portion of the attachment stem may be formed of an elastomer material. The elastomer material may be configured to deform to removably couple the syringe interface device with the plunger.

Alternatively, the attachment stem may be formed to include a first attachment portion and a second attachment portion. The first attachment portion may have a tab extending radially away from the first attachment portion and into a tab receiver formed in the second attachment portion to couple the first attachment portion and the second attachment portion together. The first attachment portion and the second attachment portion of the attachment stem may cooperate to form the hole size to receive the plunger therein. The first attachment portion and the second attachment portion may be coupled together to removably couple the syringe interface device with the plunger.

Additionally, the attachment stem may include a first attachment arm extending downwardly from the body and a second attachment arm radially spaced apart from the first attachment arm and extending downwardly from the body. A tensioning unit may wrap around the first attachment arm and the second attachment arm to couple the first attachment arm and the second attachment arm to the plunger. The tensioning unit may be located radially outward of the first attachment arm and the second attachment arm.

Optionally, the attachment stem may include a first attachment arm extending downwardly from the body and a second attachment arm radially spaced apart from the first attachment arm and extending downwardly from the body. The first attachment arm and the second attachment arm may be configured to wrap around the plunger to removably couple the syringe interface device with the plunger.

Additional features, which alone or in combination with any other feature(s), such as those listed above and/or those listed in the claims, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The foregoing and other features of the various embodiments of the methods and apparatuses described herein will become more apparent from the following detailed description and the accompanying drawings in which:

FIG. 1A is a perspective view of an adaptive syringe plunger assembly including a syringe and a syringe interface device configured to be removably coupled with the syringe, the syringe interface device having a triangle shaped body and an attachment stem extending downwardly from the body to couple with a plunger included in the syringe;

FIG. 1B is an exploded perspective view of the adaptive syringe plunger assembly of FIG. 1A showing that the syringe interface device provides a larger surface area for a syringe user to apply force while administering medications from the syringe as compared to a thumb pad of the plunger, and further suggesting that the attachment stem is configured to deform as the thumb pad of the plunger is inserted into the attachment stem and, after insertion, the thumb pad is located inside of the body of the syringe interface device;

FIG. 1C is a cross-sectional view of a portion of the adaptive syringe plunger assembly of FIG. 1A showing that the thumb pad of the plunger rests on a bottom wall of the body of the syringe interface device after the thumb pad of the plunger is inserted into the attachment stem so that the syringe interface device is retained on the syringe;

FIG. 2A is a perspective view of another embodiment of a syringe interface device configured to be removably coupled with the syringe, the syringe interface device having a conical shaped body and an attachment stem extending downwardly from the body to couple with the plunger;

FIG. 2B is an exploded perspective view of the adaptive syringe plunger assembly of FIG. 2A showing that the syringe interface device provides a larger surface area for the syringe user to apply force while administering medications from the syringe as compared to the thumb pad of the plunger, and further showing that the attachment stem is threaded so that the syringe interface device can be twisted onto the thumb pad of the plunger to retain the syringe interface device on the syringe;

FIG. 3A is a perspective view of another embodiment of a syringe interface device configured to be removably coupled with the syringe, the syringe interface device having a cylindrical shaped body and an attachment stem extending downwardly from the body to couple with the plunger;

FIG. 3B is a bottom plan view of the syringe interface device of FIG. 3A showing that the attachment stem includes a first attachment portion and a second attachment portion that snap together around the plunger, the first attachment portion having tabs that extend toward the second attachment portion to be received by tab receivers formed in the second attachment portion;

FIG. 3C is an exploded perspective view of the syringe interface device of FIG. 3A showing that the second attachment portion is coupled with the body of the syringe interface device, and further suggesting that the first attachment portion and the second attachment portion are snapped together around the plunger while the thumb pad of the plunger is inside of the body so that the syringe interface device is retained on the syringe;

FIG. 4A is a front perspective view of the syringe interface device of FIG. 3A showing that a side wall of the body is formed to include an opening to receive a hook-and-loop fastener strap therein that extends through the body and out of an opening formed on an opposing side of the side wall, the fastener strap is configured to wrap around a hand of the syringe user to support the hand of the syringe user relative to the syringe interface device during administration of the syringe;

FIG. 4B is a side perspective view of the syringe interface device of FIG. 4A showing that the fastener strap extends through the body and out of both openings formed in the side wall of the body;

FIG. 5A is a perspective view of another embodiment of a syringe interface device configured to be removably coupled with the syringe, the syringe interface device having a cylindrical shaped body and an attachment stem extending downwardly from the body to couple with the plunger, the cylindrical shaped body being formed to include a concave sidewall sized to receive fingers of the syringe user therein for the syringe user to grip, and further showing that the syringe interface device includes a tightening unit coupled with the attachment stem and having a tightening nob and a tightening wire that constricts the attachment stem after the thumb pad of the plunger has been inserted into the attachment stem so that the syringe interface device is retained on the syringe;

FIG. 5B is a perspective view of the syringe interface device of FIG. 5A showing an alternative tightening unit coupled with the attachment stem, the alternative tightening unit having a tightening hoop and a fastener configured to tighten the hoop around the attachment stem after the thumb pad of the plunger has been inserted into the attachment stem so that the syringe interface device is retained on the syringe;

FIG. 6 is a perspective view of another embodiment of a syringe interface device configured to be removably coupled with the syringe, the syringe interface device having a bent top wall and a bottom wall that form a finger-receiving region therebetween, and the bent top wall is formed to include finger-receiving grooves to receive fingers of the syringe user;

FIG. 7A is a perspective view of another embodiment of a syringe interface device configured to be removably coupled with the syringe, the syringe interface device including a hook-and-loop fastener strap formed with a slit extending therethrough and sized to receive the thumb pad of the plunger so that the syringe interface device is retained on the syringe, and further suggesting that the fastener strap is configured to wrap around the hand of the syringe user to support the hand of the syringe user relative to the syringe interface device during administration of the syringe;

FIG. 7B is a top plan view of the syringe interface device showing that the plunger extends through the slit formed in the fastener strap to retain the syringe interface device on the syringe;

FIG. 8A is a perspective view of another embodiment of a syringe interface device configured to be removably coupled with the syringe, the syringe interface device having a body and an attachment stem extending downwardly from the body to couple with the thumb pad of the plunger, the body being formed as a handle that includes finger-receiving grooves sized to receive fingers of the syringe user therein and the body being configured to move relative to the attachment stem between a vertical configuration parallel to the plunger, as shown in FIG. 8A, and a horizontal configuration, as shown in FIG. 8B, and further showing that the syringe interface device includes a body-adjustment joint formed as a ball-and-socket joint configured to allow for movement of the body between the vertical configuration and the horizontal configuration;

FIG. 8B is a perspective view of the syringe interface device of FIG. 8A in the horizontal configuration showing that a ball of the body-adjustment joint is coupled with a ball-receiver of the body-adjustment joint for rotational movement within the ball-receiver to adjust the configuration of the body relative to the plunger;

FIG. 9 is a perspective view of another embodiment of a syringe interface device configured to be removably coupled with the syringe, the syringe interface device having a body formed as a handle having grip cavities thereon and an attachment stem extending downwardly from the body to couple with the plunger, the syringe interface device further including a first coupling element coupled with a bottom surface of the attachment stem and a second coupling element coupled with a top surface of the thumb pad of the plunger, the first coupling element and the second coupling element engage one another so that the syringe interface device is retained on the syringe;

FIG. 10 is a perspective view of another embodiment of a syringe interface device configured to be removably coupled with the syringe, the syringe interface device having a body in the form of a disk and an attachment stem extending downwardly from the body to couple with the plunger, the attachment stem including a first attachment arm and a second attachment arm that each extend downwardly from opposite sides of the body;

FIG. 11 is a perspective view of the syringe interface device of FIG. 10 coupled with the plunger showing that the first attachment arm and the second attachment are configured to move inwardly toward the plunger, and further showing that the syringe interface device includes a tensioning unit that extends around the first and second attachment arms to couple the attachment arms to the plunger so that the syringe interface device is retained on the syringe; and

FIG. 12 is a perspective view of the syringe interface device of FIG. 10 coupled with the plunger showing that the first attachment arm and the second attachment are configured to wrap around the plunger so that the syringe interface device is retained on the syringe.

### DETAILED DESCRIPTION

The present disclosure relates to an adaptive syringe plunger assembly 10 that increases a surface area for a syringe user to apply force while ejecting fluid from a syringe 12. Generally, standard plungers have a small, circular end that the syringe user applies a force to through their thumb. Individuals with lower dexterity or hindered movement of their thumb may have difficulties administering syringes through such a small surface area. Thus, the small, circular end of standard plungers may lead to difficulties while administering medication for syringe users, such as clinical staff and individuals using syringes in at-home treatment.

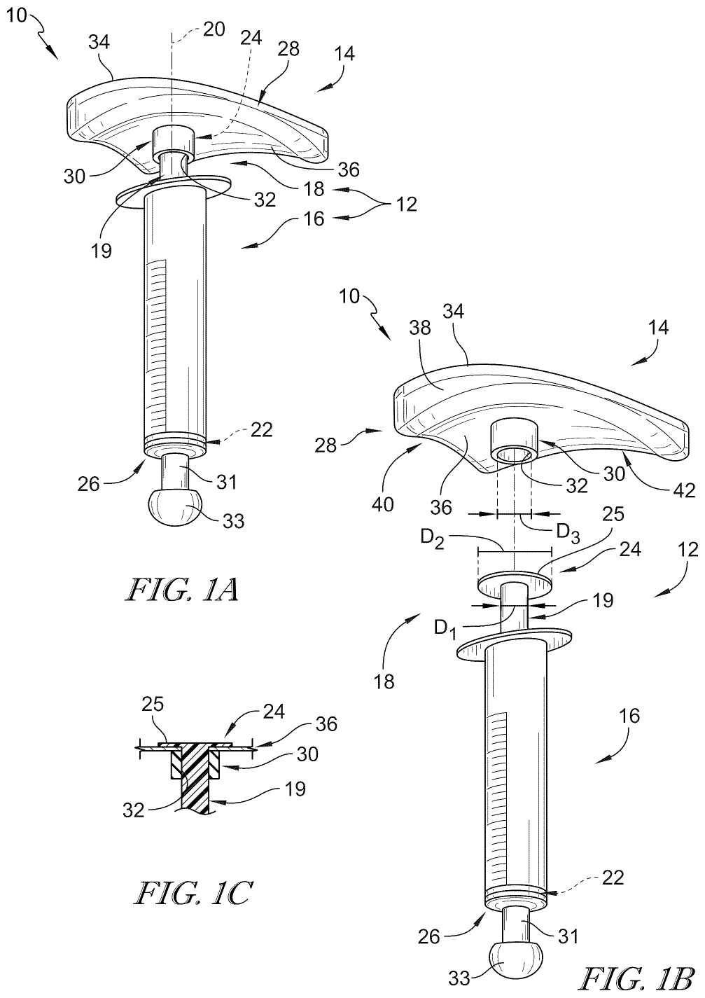

The adaptive syringe plunger assembly 10 includes the syringe 12 and at least one syringe interface device 14 as shown in FIGS. 1A and 1B. The syringe 12 is configured to eject fluid. The syringe interface device 14 has a relatively larger surface area for the syringe user to apply a force as compared to a standard plunger, and thus, less pressure is applied to the fingers of the syringe user as the force is spread over the larger surface area, such as the palm of the hand.

In the illustrative example of FIGS. 1A and 1B, the syringe 12 is an EXACTAMED™ Oral Dispenser available from Baxter International Inc. of Deerfield, Illinois, U.S.A. Thus, the syringe 12 has an open-ended tip 31 that, upon delivery, has a tip cap 33 to close the open end (not shown) of the tip 31. However, in other embodiments, a needle or leur lock end for connection to IV lines or ports, for example, is included in the syringe 12 in lieu of the tip 31.

The syringe 12 includes a syringe body 16 and a plunger 18 spaced radially inward from the syringe body 16 relative to a plunger axis 20 as shown in FIG. 1A. The syringe body 16 is hollow. The plunger 18 is received, at least partially, inside of the syringe body 16 and slides within the syringe body 16 along the plunger axis 20. The plunger 18 has a plunger shaft 19, a first end 22, and a second end 24. The plunger shaft 19 extends between and interconnects the first end 22 and the second end 24. The first end 22 of the plunger 18 is arranged inside of the syringe body 16. The second end 24 of the plunger 18 is opposite the first end 22 and is arranged outside of the syringe body 16 as shown in FIG. 1B. Illustratively, the second end 24 of the plunger 18 includes a thumb pad 25. The first end 22 of the plunger 18 is configured as a piston with a seal that slidably engages an inner surface of the syringe body 16.

As a force is applied to the syringe interface device 14 by the syringe user, the plunger 18 slides within the syringe body 16 until the first end 22 of the plunger 18 abuts a bottom end 26 of the syringe body 16 as shown in FIGS. 1A and 1B. The movement of the plunger 18 within the syringe body 16 ejects fluid from the syringe body 16. In some embodiments, the syringe 12 is used for oral administration of fluid. In some embodiments, the syringe 12 includes a needle for injecting fluid, as noted above.

At least a portion of the plunger shaft 19 of the plunger 18 is arranged outside of the syringe body 16 as shown in FIG. 1B. The portion of the plunger shaft 19 arranged outside of the syringe body 16 has a first diameter D1. The second end 24 (i.e., the thumb pad 25) of the plunger 18 has a second diameter D2 that is greater than the first diameter D1 of the plunger shaft 19.

The syringe interface device 14 is couplable with the second end 24 of the plunger 18 such that the syringe interface device 14 may be attached, removed, and/or replaced with a different syringe interface device, such as one of syringe interface devices 214, 314, 414, 514, 614, 714, 814, 914, which will be discussed in more detail below. The syringe user applies force to the syringe interface device 14 instead of applying force directly to the second end 24 of the plunger 18, which allows the force to be spread over the larger surface area. Different embodiments of the syringe interface device 14, 214, 314, 414, 514, 614, 714, 814, 914 are shown in FIGS. 1A-12. Each of the syringe interface devices 14, 214, 314, 414, 514, 614, 714, 814, 914 disclosed herein allows for the force applied by the syringe user to be spread over a larger surface area as the syringe user applies the force to the syringe interface device 14, 214, 314, 414, 514, 614, 714, 814, 914 instead of the second end 24 of the plunger 18 so that less pressure is applied to the thumb of the syringe user. That is, the syringe interface devices 14, 214, 314, 414, 514, 614, 714, 814, 914 are each larger than the thumb pad 25, thereby enhancing the ability of syringe users with low manual dexterity to administer medication, for example, with the syringe 12.

The syringe interface device 14 includes a body 28 and an attachment stem 30 as shown in FIGS. 1A and 1B. The body 28 is hollow so that the body 28 can receive the second end 24 of the plunger 18 therein as suggested in FIG. 1A and as shown in FIG. 1C. The attachment stem 30 extends downwardly from the body 28 and is formed to include a hole 32 extending through the attachment stem 30 and opening into the body 28. The hole 32 is aligned with the plunger axis 20.

The body 28 includes a top wall 34, a bottom wall 36, a first side wall 38, a second side wall 40, and a third side wall 42 as shown in FIG. 1B. A hollow space is defined between the top wall 34 and the bottom wall 36. The attachment stem 30 is coupled with the bottom wall 36. The side walls 38, 40, 42 extend between and interconnect the top wall 34 and the bottom wall 36. Illustratively, the body 28 is generally triangular in shape having three rounded points circumferentially spaced apart from one another relative to the plunger axis 20. The top wall 34 and the bottom wall 36 curve downwardly at each of the three points. An apex (i.e., a topmost point) of the top wall 34 is aligned with the plunger axis 20, and the three points of the body 28 curve downwardly toward the syringe 12. The body 28, therefore, somewhat resembles a bicycle seat and is sized to fit within a palm of the syringe user's hand. An upper surface of the top wall 34 has a surface area that is at least three times, and up to seven times or more, the surface area of the thumb pad 25.

In some embodiments, the attachment stem 30 is formed of an elastomer material. In some embodiments, the attachment stem 30 is formed of a material that allows the attachment stem 30 to deform. The hole 32 of the attachment stem 30 has a third diameter D3 that is the same as the first diameter D1 of the plunger shaft 19 as shown in FIG. 1B. To couple the plunger 18 with the syringe interface device 14, the second end 24 of the plunger 18 is inserted and pushed through the hole 32 of the attachment stem 30. The material of the attachment stem 30 allows the attachment stem 30 to deform such that the second end 24 of the plunger 18 passes through the attachment stem 30 and into the hollow space of the body 28. After the second end 24 of the plunger 18 is pushed through the hole 32 of the attachment stem 30, the second end 24 of the plunger 18 rests on top of the bottom wall 36 of the body 28 as shown in FIG. 1C. The syringe user applies force to the top wall 34 of the body 28 using, for example, the palm of their hand. During movement of the syringe interface device 14 and the plunger 18 to eject fluid, the thumb pad 25 may separate away from the bottom wall 36 until the top wall 34 of the body 28 engages an upper surface of the thumb pad 25.

While the syringe interface device 14 and the syringe 12 are coupled together, the attachment stem 30 is located radially outward of the plunger shaft 19 as shown in FIG. 1C. Because the second diameter D2 of the second end 24 of the plunger 18 is greater than the third diameter D3 of the hole 32, the second end 24 of the plunger 18 is retained in the hollow space of the body 28 so that the syringe interface device 14 remains attached to the syringe 12. The second end 24 of the plunger 18 can be removed from the hole 32 through force great enough to deform the material of the attachment stem 30.

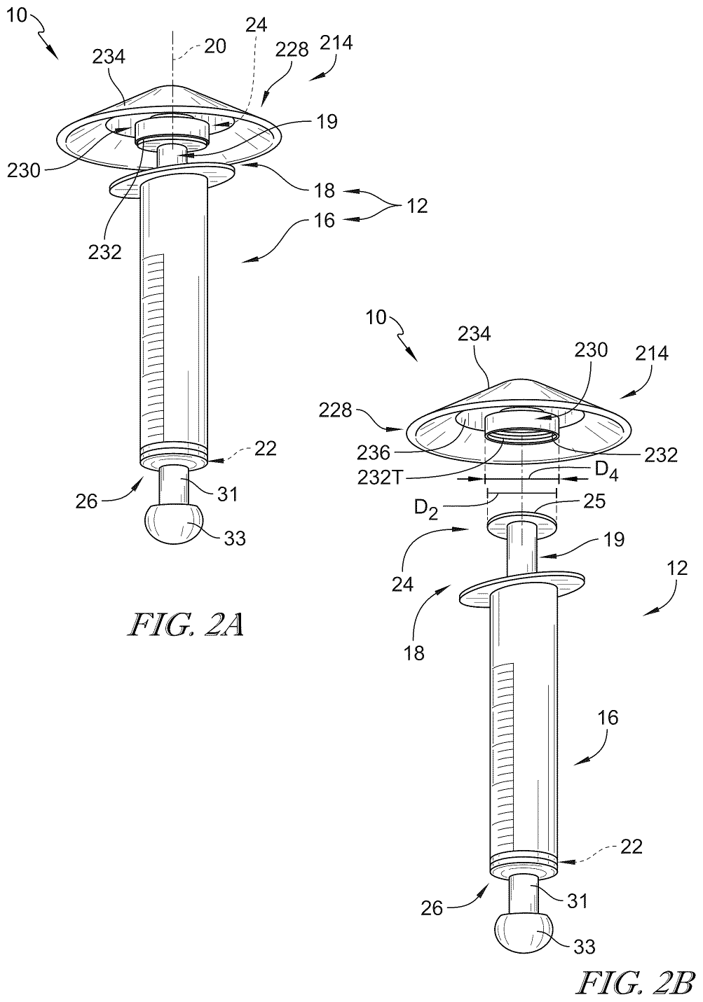

Another syringe interface device 214 couplable with the second end 24 of the plunger 18 is shown in FIGS. 2A and 2B. The syringe interface device 214 includes a body 228 and an attachment stem 230.

The body 228 includes a top wall 234 and a bottom wall 236 as shown in FIG. 2B. A hollow space is defined between a portion of the top wall 234 and the bottom wall 236. The top wall 234 is formed to include an apex aligned with the plunger axis 20. The top wall 234 is angled downwardly from the apex relative to the plunger axis 20. Illustratively, the top wall 234 is conical in shape. The bottom wall 236 is arranged radially inward of the top wall 234 relative to the plunger axis 20 to define the hollow space between the portion of the top wall 234 and the bottom wall 236. The bottom wall 236 extends substantially perpendicular to the plunger axis 20.

The attachment stem 230 is coupled with the bottom wall 236 and extends downwardly from the bottom wall 236 as shown in FIGS. 2A and 2B. The attachment stem 230 is formed to include a hole 232 extending through the attachment stem 230. The hole 232 is aligned with the plunger axis 20.

In some embodiments, the hole 232 of the attachment stem 230 is formed to include threads 232T as shown in FIG. 2B. The hole 232 of the attachment stem 230 has a fourth diameter D4 that is substantially similar to the second diameter D2 of the second end 24 of the plunger 18 as shown in FIG. 2B. The threads 232T of the hole 232 engage the second end 24 of the plunger 18 as the syringe interface device 214 is twisted or rotated onto the second end 24 of the plunger 18. After the syringe interface device 214 is twisted onto the second end 24 of the plunger 18, the second end 24 of the plunger 18 is retained within the hole 232 through engagement with the threads 232T so that the syringe interface device 214 remains attached to the syringe 12. The syringe user applies force to the top wall 234 of the body 228 using, for example, the palm of their hand. The syringe interface device 214 can be removed through twisting of the syringe interface device 214 in an opposite direction as that used during the coupling. An upper surface of the top wall 234 has a surface area that is at least three times, and up to seven times or more, the surface area of the thumb pad 25.

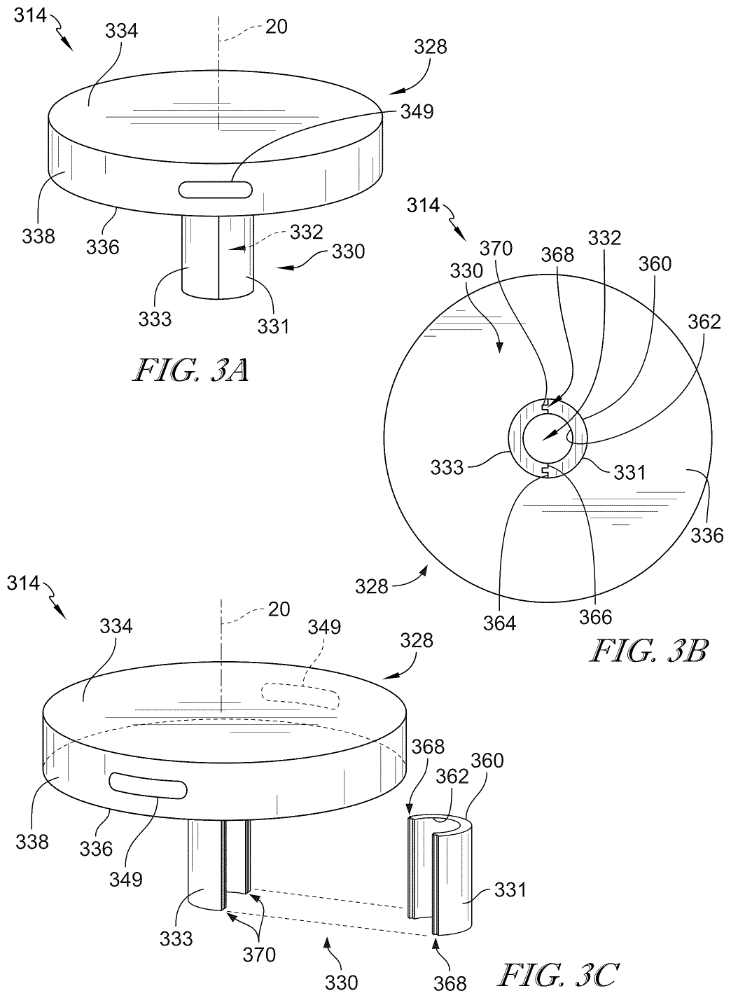

Another syringe interface device 314 couplable with the second end 24 of the plunger 18 is shown in FIGS. 3A-3C. The syringe interface device 314 includes a body 328 and an attachment stem 330. The body 328 is hollow so that the body 328 can receive the second end 24 of the plunger 18 therein. The attachment stem 330 extends downwardly from the body 328 and is formed to include a hole 332 extending through the attachment stem 330 and opening into the body 328. The hole 332 is aligned with the plunger axis 20.

The body 328 includes a top wall 334, a bottom wall 336, and a side wall 338 as shown in FIG. 3A. A hollow space is defined between the top wall 334 and the bottom wall 336. The side wall 338 extends around the plunger axis 20 and interconnects the top wall 334 and the bottom wall 336. Illustratively, the body 328 is cylindrical in shape having a substantially flat top wall 334 and a substantially flat bottom wall 336 relative to the plunger axis 20. The top wall 334 and the bottom wall 336 are substantially perpendicular to the plunger axis 20 and are substantially parallel to one another. Though the body 328 is shown and described as a cylindrical shape, other shapes that increase a surface area for the syringe user to apply a force are contemplated.

In some embodiments, the attachment stem 330 is formed of a plastics material. The attachment stem 330 includes a first attachment portion 331 and a second attachment portion 333 as shown in FIG. 3B. The first attachment portion 331 and the second attachment portion 333 fit together around the plunger shaft 19 to couple the syringe interface device 314 with the syringe 12. The first attachment portion 331 and the second attachment portion 333 are each illustratively formed as half of a hollow cylinder. In alternative embodiments, the first attachment portion 331 and the second attachment portion 333 are each formed as half of a hollow rectangular prism, for example.

Each of the first attachment portion 331 and the second attachment portion 333 includes an outer surface 360 and an inner surface 362 spaced radially inward of the outer surface 360 relative to the plunger axis 20 as shown in FIGS. 3B and 3C. The first attachment portion 331 includes two first connector faces 364 that face toward the second attachment portion 333 and interconnect the outer surface 360 and the inner surface 362 of the first attachment portion 331. The second attachment portion 333 includes two second connector faces 366 that face toward the first attachment portion 331 and interconnect the outer surface 360 and the inner surface 362 of the second attachment portion 333.

The first attachment portion 331 includes a tab 368 extending outwardly from each of the first connector faces 364 as shown in FIGS. 3B and 3C. The second attachment portion 333 includes a tab receiver 370 extending inwardly from each of the second connector faces 366. Each of the tab receivers 370 is sized to receive a corresponding tab 368 to couple the first attachment portion 331 and the second attachment portion 333 together as shown in FIG. 3B.

While the first attachment portion 331 and the second attachment portion 333 are coupled together, the inner surfaces 362 of the first attachment portion 331 and the second attachment portion 333 cooperate to form the hole 332 of the attachment stem 330 as shown in FIG. 3B. A diameter of the hole 332 is substantially equivalent to the first diameter D1 of the plunger shaft 19. To couple the syringe interface device 314 to the syringe 12, the plunger shaft 19 is placed adjacent the inner surface 362 of the second attachment portion 333 so that the second end 24 of the plunger 18 is located within the hollow space of the body 328. The first attachment portion 331 is moved toward the second attachment portion 333 until each of the tabs 368 snap into engagement with the corresponding tab receiver 370. Because the second end 24 of the plunger 18 is located within the hollow space of the body 328 on top of the bottom wall 336, and the plunger shaft 19 is located radially inward of the attachment portions 331, 333, the syringe interface device 314 remains attached to the syringe 12. The syringe user applies a force to the top wall 334 of the body 328 using, for example, the palm of their hand. An upper surface of the top wall 334 has a surface area that is at least three times, and up to seven times or more, the surface area of the thumb pad 25. The attachment portions 331, 333 can be snapped apart to remove the syringe interface device 314 from the syringe 12.

In some embodiments, as shown in FIG. 3C, the second attachment portion 333 is fixed to the bottom wall 336 of the body 328, and the first attachment portion 331 is not fixed to the bottom wall 336 of the body 328. In some embodiments, the body 328 is split in half so that a first portion of the body 328 is fixed to the first attachment portion 331 and a second portion of the body 328 is fixed to the second attachment portion 333.

As shown in FIG. 3C, the tabs 368 and the tab receivers 370 extend along an entirety of a height of the attachment portions 331, 333, respectively. In alternative embodiments, the tabs 368 and the tab receivers 370 extend along a portion of the height of the attachment portions 331, 333, respectively.

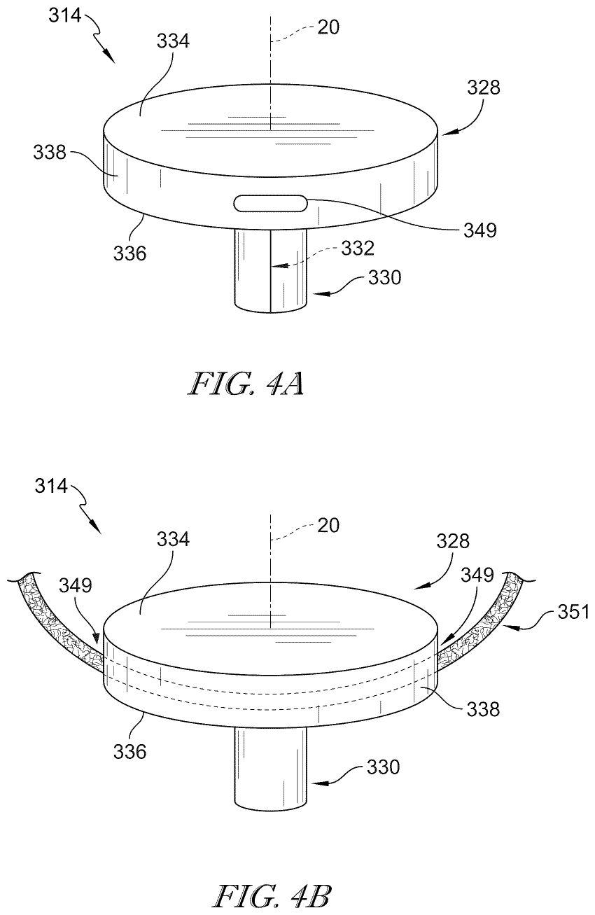

In some embodiments, the syringe interface device 314 further includes a hook-and-loop fastener strap 351, as shown in FIGS. 4A and 4B, to hold the syringe interface device 314 to the hand of the syringe user. In such an embodiment, the side wall 338 of the body 328 is formed to include two openings 349 on opposing sides of the body 328 as shown best in FIG. 3C. The openings 349 each open into the hollow space of the body 328. The fastener strap 351 extends through one opening 349, through the hollow space of the body 328, and out of the other opening 349. The syringe user can place their palm on the top wall 334 of the body 328 and wrap the fastener strap 351 around their hand. The fastener strap 351 is adjustable to fit the size of the hand of the syringe user. The fastener strap 351 couples the hand of the syringe user to the syringe interface device 314, and thus, the syringe 12. The fastener strap 351 allows for increased grip by the syringe user.

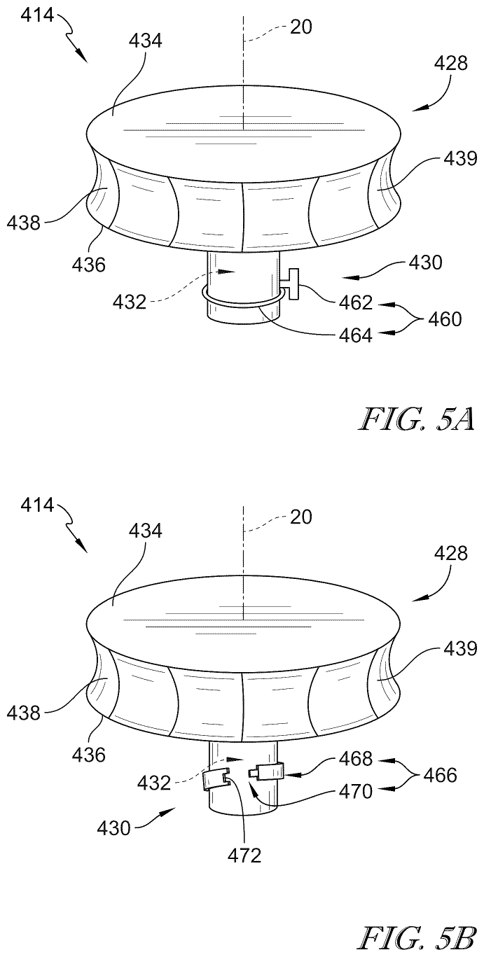

Another syringe interface device 414 couplable with the second end 24 of the plunger 18 is shown in FIGS. 5A and 5B. The syringe interface device 414 includes a body 428 and an attachment stem 430. The attachment stem 430 extends downwardly from the body 428 and is formed to include a hole 432 extending through the attachment stem 430. The hole 432 is aligned with the plunger axis 20.

The body 428 includes a top wall 434, a bottom wall 436, and a concave side wall 438 as shown in FIGS. 5A and 5B. A hollow space is defined between the top wall 434 and the bottom wall 436. The attachment stem 430 is coupled with the bottom wall 436. The side wall 438 extends around the plunger axis 20 and interconnects the top wall 434 and the bottom wall 436. Illustratively, the body 428 is cylindrical in shape having a substantially flat top wall 434 and a substantially flat bottom wall 436 relative to the plunger axis 20. The top wall 434 and the bottom wall 436 are substantially perpendicular to the plunger axis 20 and substantially parallel to one another. The side wall 438 is concave and curves radially inwardly toward the plunger axis 20 to form a toroidal notch 439 extending circumferentially around the side wall 438. The notch 439 is sized to receive fingers of the syringe user therein to allow for increased grip by the syringe user, if the syringe user decides to drip the syringe interface device 414 in that manner.

In some embodiments, the attachment stem 430 is formed of an elastomer material. In some embodiments, the attachment stem 430 is formed of a material that allows the attachment stem 430 to deform. The hole 432 of the attachment stem 430 has a diameter that is substantially similar to the second diameter D2 of the second end 24 of the plunger 18.

In some embodiments, the syringe interface device 414 further includes a tightening unit 460 as shown in FIG. 5A. The tightening unit 460 includes a tightening nob 462 and a tightening wire 464. The tightening nob 462 is coupled with the attachment stem 430 and is configured to rotate relative to the attachment stem 430. The tightening wire 464 is coupled with the tightening nob 462 and extends around the attachment stem 430. The tightening wire 464 winds and unwinds around the tightening nob 462 as the tightening nob 462 rotates, depending on a direction of rotation. Both ends of the tightening wire 464 are coupled with the tightening nob 462, and the tightening wire 464 loosely extends around the attachment stem 430 as shown in FIG. 5A.

To couple the syringe interface device 414 with the syringe 12, the second end 24 of the plunger 18 is inserted into the hole 432 of the attachment stem 430. The second end 24 of the plunger 18 remains in the hole 432 of the attachment stem 430. The hole 432 has a similar diameter to the second diameter D2 of the second end 24 of the plunger 18, so the tightening unit 460 retains the second end 24 of the plunger 18 within the hole 432. In response to the tightening nob 462 being rotated while the second end 24 of the plunger 18 is located within the hole 432, the tightening wire 464 tightens around the attachment stem 430 as the tightening wire 464 winds around the tightening nob 462. The tightening wire 464 constricts the attachment stem 430 below the second end 24 of the plunger 18 until the tightening wire 464 has tightened sufficiently around the attachment stem 430 (and the plunger shaft 19 inside of the attachment stem 430). The tightening wire 464 retains the second end 24 of the plunger 18 above the tightening wire 464. The syringe user applies force to the top wall 434 of the body 428 using, for example, the palm of their hand. An upper surface of the top wall 434 has a surface area that is at least three times, and up to seven times or more, the surface area of the thumb pad 25.

To remove the syringe interface device 414 from the syringe 12, the tightening nob 462 is rotated in an opposite direction. As the tightening nob 462 is rotated in the opposite direction, the tightening wire 464 unwinds from the tightening nob 462 and loosens around the attachment stem 430 so that the plunger 18 can be removed from the hole 432.

In some embodiments, the syringe interface device 414 includes an alternative tightening unit 466, as shown in FIG. 5B, instead of the tightening unit 460. The tightening unit 466 includes a tightening hoop 468 and a fastener 470. The tightening hoop 468 is coupled with the attachment stem 430 and is configured to tighten around the attachment stem 430. The tightening hoop 468 includes a first end and a second end. The fastener 470 is coupled with and extends from the first end of the tightening hoop 468. The second end of the tightening hoop 468 is formed to include a fastener receiver 472 sized to receive the fastener 470 therein to tighten the tightening hoop 468 around the attachment stem 430. While the fastener 470 is not coupled with the fastener receiver 472, as shown in FIG. 5B, the tightening hoop 468 does not form a full hoop around the attachment stem 430.

To couple the syringe interface device 414 with the syringe 12, the second end 24 of the plunger 18 is inserted into the hole 432 of the attachment stem 430. The second end 24 of the plunger 18 remains in the hole 432 of the attachment stem 430. The hole 432 has a similar diameter to the second diameter D2 of the second end 24 of the plunger 18, so the tightening unit 466 retains the second end 24 of the plunger 18 within the hole 432. After the second end 24 of the plunger 18 is inserted into the hole 432, the fastener 470 is coupled with the fastener receiver 472 to tighten the tightening hoop 468 around the attachment stem 430.

The tightening hoop 468 constricts the attachment stem 430 below the second end 24 of the plunger 18 until the tightening hoop 468 has tightened sufficiently around the attachment stem 430 (and the plunger shaft 19 inside of the attachment stem 430). The tightening hoop 468 retains the second end 24 of the plunger 18 above the tightening hoop 468. The syringe user applies force to the top wall 434 of the body 428 using, for example, the palm of their hand. To remove the syringe interface device 414 from the syringe 12, the fastener 470 is decoupled from the fastener receiver 472, which loosens the tightening hoop 468 around the attachment stem 430.

In some embodiments, the tightening hoop 468 is formed of a plastics material. In some embodiments, the tightening hoop 468 is formed of an elastomer material. In some embodiments, the tightening hoop 486 is made of a resilient or stretchable material with a middle region being adhered or otherwise fastened to the attachment stem 430 to retain the tightening hoop 468 thereon when the fastener 470 is not fastened to the fastener receiver 472 as shown in FIG. 5B. In some embodiments, the fastener 470 is a screw with threads and the fastener receiver 472 is formed to include threads that mate with the threads of the fastener 470.

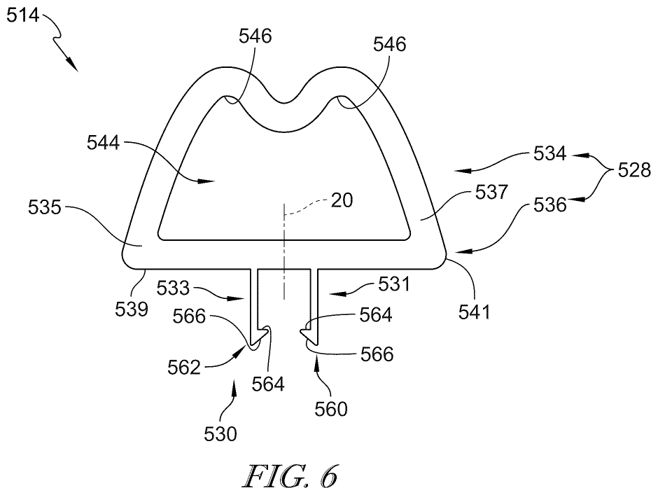

Another syringe interface device 514 couplable with the second end 24 of the plunger 18 is shown in FIG. 6. The syringe interface device 514 includes a body 528 and an attachment stem 530. The body 528 includes a top wall 534 and a bottom wall 536. The top wall 534 of the body 528 is somewhat M-shaped and is coupled with and extends upwardly from opposing edges of the bottom wall 536. The attachment stem 530 extends downwardly from the bottom wall 536.

The top wall 534 extends between a first end 535 and a second end 537 as shown in FIG. 6. The first end 535 of the top wall 534 is coupled with a first end 539 of the bottom wall 536, and the second end 537 of the top wall 534 is coupled with a second end 541 of the bottom wall 536 opposite the first end 539 of the bottom wall 536. The first end 535 of the top wall 534 extends upwardly away from the first end 539 of the bottom wall 536, and the second end 537 of the top wall 534 extends upwardly away from the second end 541 of the bottom wall 536 to form a finger-receiving region 544 between the top wall 534 and the bottom wall 536.

The top wall 534 is bent between the first end 535 and the second end 537 to form finger-receiving grooves 546 as shown in FIG. 6. Fingers of the syringe user wrap under the top wall 534 and engage the finger-receiving grooves 546 to apply force using, for example, a portion of their palm and multiple fingers. Though illustrated with two finger-receiving grooves 546, any number of finger-receiving grooves 546 is contemplated, such as, but not limited to, one groove, two grooves, three grooves, or four grooves.

The bottom wall 536 is substantially perpendicular to the plunger axis 20 as shown in FIG. 6. In some embodiments, the bottom wall 536 is formed as an annular disk extending about the plunger axis 20. In alternative embodiments, the bottom wall 536 is formed as a rectangular prism. In some embodiments, the body 528 is formed as a single integral component.

In some embodiments, the attachment stem 530 is formed of a plastics material. In the illustrative embodiment, the attachment stem 530 includes a first attachment arm 531 and a second attachment arm 533 as shown in FIG. 6. The first attachment arm 531 and the second attachment arm 533 are radially spaced apart from one another so that the plunger 18 is received radially between the first attachment arm 531 and the second attachment arm 533. A first tab 560 is coupled with a bottom end of the first attachment arm 531. A second tab 562 is coupled with a bottom end of the second attachment arm 533. Each of the first tab 560 and the second tab 562 extends radially inwardly toward the plunger axis 20 and toward one another. A distance between the first tab 560 and the second tab 562 is less than the second diameter D2 of the second end 24 of the plunger 18. The tabs 560, 562 snap under the second end 24 of the plunger 18 as the second end 24 of the plunger 18 is inserted into the syringe interface device 514 between the first attachment arm 531 and the second attachment arm 533. As the second end 24 of the plunger 18 is inserted between the first attachment arm 531 and the second attachment arm 533, the tabs 560, 562 flex outwardly to allow the second end 24 of the plunger 18 to move past the tabs 560, 562. The second end 24 of the plunger 18 rests on top of the tabs 560, 562 so that the syringe interface device 514 remains attached to the syringe 12.

Each tab 560, 562 includes a top surface 564 and an angled surface 566 as shown in FIG. 6. The top surfaces 564 face toward the bottom wall 536 of the body 528 and engage the second end 24 of the plunger 18 after the plunger 18 is inserted into the syringe interface device 514. The top surfaces 564 are substantially perpendicular to the plunger axis 20. The angled surfaces 566 are angled relative to the plunger axis 20 as shown in FIG. 6. The angled surfaces 566 engage the second end 24 of the plunger 18 while the second end 24 is inserted into the syringe interface device 514. The second end 24 of the plunger 18 is moved upwardly into engagement with the angled surfaces 566 of the tabs 560, 562, which pushes the tabs 560, 562 radially outward so that the second end 24 of the plunger 18 can be inserted into the syringe interface device 514. To remove the syringe 12 from the syringe interface device 514, the tabs 560, 562 move radially outwardly away from one another so that there is clearance for the second end 24 of the plunger 18 to be removed from the syringe interface device 514.

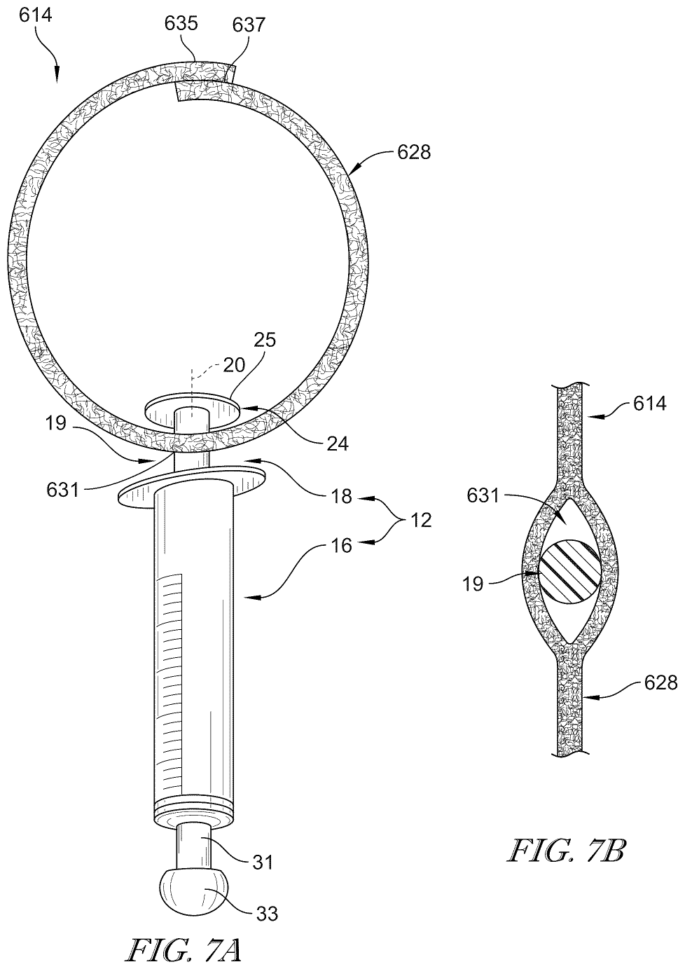

Another syringe interface device 614 couplable with the second end 24 of the plunger 18 is shown in FIGS. 7A and 7B. The syringe interface device 614 includes a body 628 formed with a slit 631 extending therethrough as shown in FIG. 7B. The slit 631 extends through the body 628 without bisecting an edge of the body 628. That is, the slit 631 is parallel with the long dimension of the body 628. Illustratively, the body 628 is a hook-and-loop fastener strap 628. The slit 631 is sized to receive the second end 24 of the plunger 18 therein. After the second end 24 of the plunger 18 is inserted through the slit 631, the fastener strap 628 is retained on the second end 24 of the plunger 18 so that the syringe interface device 14 remains attached to the syringe 12. The fastener strap 628 wraps around the hand of the syringe user, and opposing ends 635, 637 of the fastener strap 628 couple with one another to secure the syringe interface device 614 to the hand of the syringe user. The fastener strap 628 is adjustable to the hand size of the syringe user as the fastener strap 628 can be tightened and loosened around the hand of the syringe user. The syringe user applies force to the second end 24 of the plunger 18 using, for example, the palm of their hand. To remove the syringe 12 from the syringe interface device 614, the second end 24 of the plunger 18 passes through the slit 631.

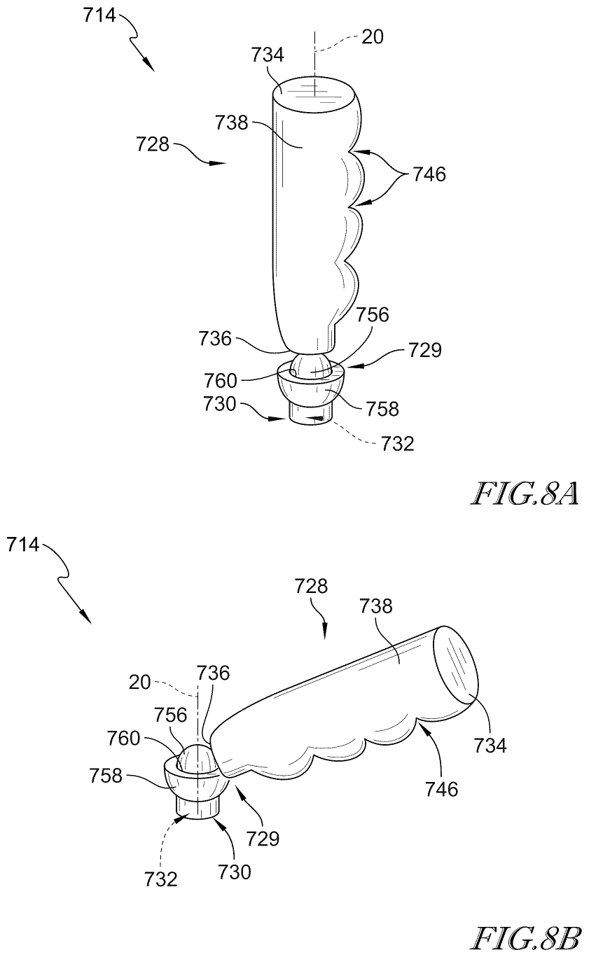

Another syringe interface device 714 couplable with the second end 24 of the plunger 18 is shown in FIGS. 8A and 8B. The syringe interface device 714 includes a body 728 in the form of a grip handle, a body-adjustment joint 729, and an attachment stem 730. An angle of the body 728 relative to the plunger axis 20 can be adjusted using the body-adjustment joint 729. For example, as shown in FIG. 8A, the body 728 extends along the plunger axis 20. As shown in FIG. 8B, the body 728 extends at an inclined angle of about 60 degrees relative the plunger axis 20. Though, the body 728 may extend at any angle relative to the plunger axis 20. Some syringe users may find the body 728 easier to grip, and thus, easier to apply force to the plunger 18, if the body 728 extends at a particular angle relative to the plunger axis 20. The syringe user may find that particular body motions and/or grip angles are easier to achieve, so the body-adjustment joint 729 enables the syringe user to adapt the syringe interface device 714 to the body motion and/or grip angle that is easiest for the syringe user. The body-adjustment joint 729 is illustratively formed as a ball-and-socket joint. The attachment stem 730 extends downwardly from the body-adjustment joint 729 to couple to the second end 24 of the plunger 18.

The body 728 includes a top wall 734, a bottom wall 736, and a side wall 738 as shown in FIGS. 8A and 8B. The side wall 738 extends between and interconnects the top wall 734 and the bottom wall 736. The body-adjustment joint 729 extends downwardly from the bottom wall 736. At least a portion of the side wall 738 is formed to include finger-receiving grooves 746 thereon as shown in FIGS. 8A and 8B. Fingers of the syringe user wrap around the side wall 738 and fit within the finger-receiving grooves 746 to apply force to the plunger 18. Though illustrated with three finger-receiving grooves 746, any number of finger-receiving grooves 746 is contemplated, such as, but not limited to, one groove, two grooves, three grooves, or four grooves.

The body-adjustment joint 729 includes a ball 756 and a ball receiver 758 formed to include a depression 760 sized to receive the ball 756 therein as shown in FIGS. 8A and 8B. The ball 756 is fixed to the bottom wall 736 so that the bottom wall 736, and thus, the body 728 moves with the ball 756. The ball 756 is coupled to the ball receiver 758 for rotational movement within the depression 760 of the ball receiver 758. The ball 756 rotates within the depression 760 to adjust the angle of the body 728 relative to the plunger axis 20. As suggested in FIGS. 8A and 8B, the syringe user grips the body 728 and moves the body 728, which causes the ball 756 to move within the depression 760 to adjust the angle of the body 728 relative to the plunger axis 20.

The attachment stem 730 extends downwardly from the ball receiver 758 and is formed to include a hole 732 extending through the attachment stem 730 to receive the second end 24 of the plunger 18 therein as shown in FIG. 8A. In some embodiments, the attachment stem 730 is formed of an elastomer material. In such an embodiment, the hole 732 of the attachment stem 730 has a diameter that is substantially similar to the first diameter D1 of the plunger shaft 19. The elastomer material of the attachment stem 730 is configured to deform such that the second end 24 of the plunger 18 passes through the attachment stem 730 and into a hollow space of the ball receiver 758. After the second end 24 of the plunger 18 is pushed through the hole 732, the second end 24 of the plunger 18 rests in the hollow space of the ball receiver 758 so that the syringe interface device 714 remains attached to the syringe 12.

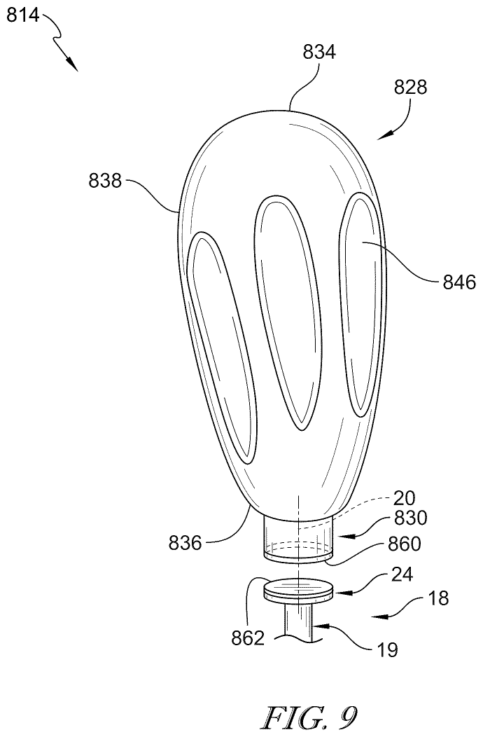

Another syringe interface device 814 couplable with the second end 24 of the plunger 18 is shown in FIG. 9. The syringe interface device 814 includes a body 828 in the form of a grip handle and an attachment stem 830. The body 828 extends along the plunger axis 20.

The body 828 includes a top wall 834, a bottom wall 836, and a side wall 838 as shown in FIG. 9. The side wall 838 extends between and interconnects the top wall 834 and the bottom wall 836. In some embodiments, the top wall 834 is substantially flat relative to the plunger axis 20 (i.e., perpendicular to the plunger axis 20). In some embodiments, the top wall 834 and the side wall 838 are formed as a continuous wall such that the top wall 834 is rounded.

At least a portion of the side wall 838 is formed to include grip cavities or depressions 846 thereon as shown in FIG. 9. Fingers of the syringe user wrap around the side wall 838 and fit within the grip cavities 846 to increase the syringe user's grip on the syringe interface device 814. In the illustrative embodiment, the grip cavities 846 extend on the side wall 838 along the plunger axis 20 partially between the top wall 834 and the bottom wall 836 (i.e., the grip cavities 846 extend parallel to the plunger axis 20). In alternative embodiments, the grip cavities 846 extend circumferentially around the side wall 838 (i.e., the grip cavities 846 extend perpendicular to the plunger axis 20). There may be any number of grip cavities 846 formed on the side wall 838.

The attachment stem 830 extends downwardly from the bottom wall 836 of the body 828 as shown in FIG. 9. In some embodiments, the syringe interface device 814 further includes a first coupling element 860 attached to a bottom surface of the attachment stem 830 and a second coupling element 862 attached to a top surface of the second end 24 of the plunger 18. The first coupling element 860 and the second coupling element 862 engage one another to retain the syringe interface device 814 on the syringe 12.

For example, in some embodiments, the coupling elements 860, 862 are hook-and-loop fasteners. One side of the hook-and-loop fastener is adhered to the bottom surface of the attachment stem 830, and the other side of the hook-and-loop fastener is adhered to the top surface of the second end 24 of the plunger 18. In response to pressing the syringe interface device 814 and the second end 24 of the plunger 18 together, the coupling elements 860, 862 fasten the syringe interface device 814 and the syringe 12 together temporarily.

In some embodiments, the coupling elements 860, 862 are magnets. One magnet is adhered to the second end 24 of the plunger 18, such as, though an adhesive. Another magnet is adhered to (or embedded in) the attachment stem 830. In response to placing the syringe interface device 814 and the second end 24 of the plunger 18 near one another, the magnets are attracted to one another to retain the syringe interface device 814 to the syringe 12 temporality. In some embodiments, the attachment stem 830 is omitted altogether such that the first coupling element 860 is adhered to or embedded in the bottom wall 836 of the body 628.

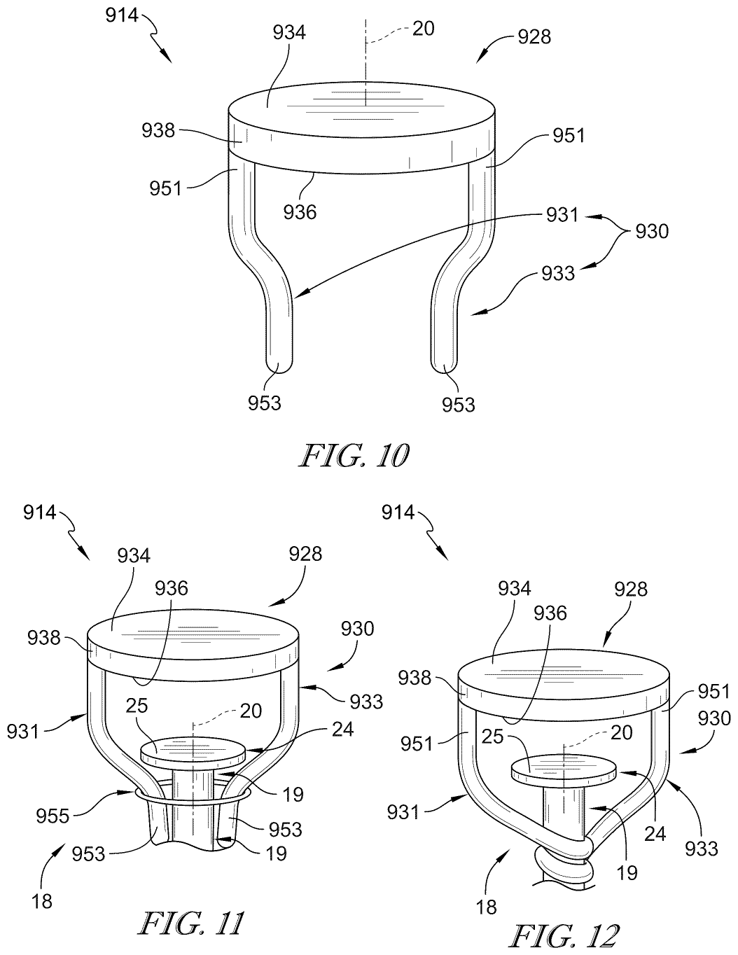

Another syringe interface device 914 couplable with the second end 24 of the plunger 18 is shown in FIGS. 10-12. The syringe interface device 914 includes a body 928 and an attachment stem 930. The attachment stem 930 extends downwardly from the body 928. In the illustrative embodiment, the attachment stem 930 includes a first attachment arm 931 and a second attachment arm 933. Though shown and described with two attachment arms 931, 933, any number of attachment arms is contemplated.

The body 928 includes top wall 934, a bottom wall 936, and a side wall 938 as shown in FIG. 10. Illustratively, the body 928 is cylindrical in shape and has a substantially flat top wall 934 and a substantially flat bottom wall 936 relative to the plunger axis 20. The top wall 934 and the bottom wall 936 are substantially perpendicular to the plunger axis 20 and parallel to one another. The side wall 938 extends around the plunger axis 20 and interconnects the top wall 934 and the bottom wall 936. The first attachment arm 931 and the second attachment arm 933 are coupled with the bottom wall 936 at opposing sides of the bottom wall 936 relative to the plunger axis 20. The first attachment arm 931 and the second attachment arm 933 are radially spaced apart from one another so that the syringe 12 is received radially between the first attachment arm 931 and the second attachment arm 933.

Though shown and described as a cylindrical shaped body 928, the body 928 may have any other shape that increases the surface area for force to be applied by the syringe user. For example, the body 928 can be shaped similarly to the body 28, the body 228, the body 428, the body 528, the body 728, or the body 828, among other shapes.

In some embodiments, the first attachment arm 931 and the second attachment arm 933 are formed of an elastomer material. In some embodiments, the first attachment arm 931 and the second attachment arm 933 are formed of rubber coated metal. The first attachment arm 931 and the second attachment arm 933 are flexible such that the first attachment arm 931 and the second attachment arm 933 can be moved toward the plunger shaft 19 as shown in FIGS. 11 and 12.

The first attachment arm 931 and the second attachment arm 933 each include a first end 951 fixed to the bottom wall 936 and a second end 953 opposite the first end 951 that is free for movement as suggested in FIG. 10. The second end 953 of each of the attachment arms 931, 933 is free to move toward and away from the plunger shaft 19.

As shown in FIG. 11, in some embodiments, the syringe interface device 914 further includes a tensioning device 955, such as, for example, an elastic loop. The tensioning device 955 couples the first attachment arm 931 and the second attachment arm 933 with the plunger shaft 19. For example, to couple the syringe interface device 914 with the syringe 12, the second end 24 of the plunger 18 is positioned below the body 928 of the syringe interface device 914 and radially between the first attachment arm 931 and the second attachment arm 933. The first attachment arm 931 and the second attachment arm 933 are moved radially inward toward each other and toward the plunger shaft 19. The tensioning device 955 loops over and around the first attachment arm 931 and the second attachment arm 933, and thus, retains the syringe interface device 914 to the syringe 12. The syringe user applies force to the top wall 934 of the body 928 using, for example, the palm of their hand. An upper surface of the top wall 934 has a surface area that is at least three times, and up to seven times or more, the surface area of the thumb pad 25.

To remove the syringe interface device 914 from the syringe 12, the tensioning device 955 is looped back over the first attachment arm 931 and the second attachment arm 933 so that it does not extend around the first attachment arm 931 and the second attachment arm 933. In some embodiments, the tensioning device 955 is completely separable from the first attachment arm 931 and the second attachment arm 933. In some embodiments, the tensioning device 955 is fixed to one of the attachment arms 931, 933. For example, in such an embodiment, one of the attachment arms 931, 933 is formed to include a through hole extending through the second end 953 of the attachment arm 931, 933. The tensioning device 955 extends through the through hole so that the tensioning device 955 remains coupled to one of the attachment arms 931, 933.

As shown in FIG. 12, in some embodiments, the attachment arms 931, 933 are configured to twist around the plunger shaft 19 of the plunger 18 to couple the syringe interface device 914 with the syringe 12. To remove the syringe interface device 914 from the syringe 12, the attachment arms 931, 933 are untwisted from the plunger shaft 19.

In some embodiments of the syringe interface device 914, the tensioning device 955 is omitted altogether. In some embodiments of the syringe interface device 914, the tensioning device 955 is included in the syringe interface device 914 and the attachment arms 931, 933 are configured to twist around the plunger shaft 19 such that the syringe user can use one or both attachment methods.

Though the syringe interface devices 14, 214, 314, 414, 514, 714, 814, 914 are each described and illustrated as having the corresponding attachment stem 30, 230, 330, 430, 530, 730, 830, 930, each of the syringe interface devices 14, 214, 314, 414, 514, 714, 814, 914 can include any of the attachment stems 30, 230, 330, 430, 530, 730, 830, 930 disclosed herein. For example, in some embodiments, the syringe interface device 14 includes any of the attachment stems 230, 330, 430, 530, 930. In some embodiments, the syringe interface device 14 includes the coupling elements 860, 862.

In some embodiments, the syringe interface device 214 includes any of the attachment stems 30, 330, 430, 530, 930. In some embodiments, the syringe interface device 214 includes the coupling elements 860, 862.

In some embodiments, the syringe interface device 314 includes any of the attachment stems 30, 230, 430, 530, 930. In some embodiments, the syringe interface device 314 includes the coupling elements 860, 862. In some embodiments, the syringe interface device 414 includes any of the attachment stems 30, 230, 330, 530, 930. In some embodiments, the syringe interface device 414 includes the coupling elements 860, 862.

In some embodiments, the syringe interface device 514 includes any of the attachment stems 30, 230, 330, 430, 930. In some embodiments, the syringe interface device 514 includes the coupling elements 860, 862. In some embodiments, the syringe interface device 714 includes any of the attachment stems 30, 230, 330, 430, 530, 930. In some embodiments, the syringe interface device 714 includes the coupling elements 860, 862.

In some embodiments, the syringe interface device 814 includes any of the attachment stems 30, 230, 330, 430, 530, 930. In some embodiments, the body 28, 228, 428, 928 of the syringe interface device 14, 214, 414, 914 is formed to include the openings 349 for the fastener strap 351 to extend through and wrap around the hand of the syringe user. In alternative embodiments, the plunger 18 and any of the syringe interface devices 14, 214, 314, 414, 514, 714, 814, 914 are manufactured as a single component so that the plunger 18 and the syringe interface devices 14, 214, 314, 414, 514, 714, 814, 914 are permanently fixed to one another.

In some embodiments, the syringe interface devices 14, 214, 314, 414, 514, 714, 814, 914 are formed of hard plastic. In some embodiments, the syringe interface devices 14, 214, 314, 414, 514, 714, 814, 914 are formed of flexible plastic. In some embodiments, the syringe interface devices 14, 214, 314, 414, 514, 714, 814, 914 are formed of rubber. In some embodiments, the syringe interface devices 14, 214, 314, 414, 514, 714, 814, 914 are formed of polymer. In some embodiments, the syringe interface devices 14, 214, 314, 414, 514, 714, 814, 914 are formed of elastomer. In some embodiments, the syringe interface devices 14, 214, 314, 414, 514, 714, 814, 914 are formed of metal.

In some embodiments, the syringe interface device 14, 214, 314, 414, 514, 714, 814, 914 and the attachment stem 30, 230, 330, 430, 530, 730, 830, 930 coupled with the syringe interface device 14, 214, 314, 414, 514, 714, 814, 914 are formed of the same material. In some embodiments, the syringe interface device 14, 214, 314, 414, 514, 714, 814, 914 and the attachment stem 30, 230, 330, 430, 530, 730, 830, 930 coupled with the syringe interface device 14, 214, 314, 414, 514, 714, 814, 914 are formed of different material.

The present disclosure contemplates that two or more syringe interface devices 14, 214, 314, 414, 514, 614, 714, 814, 914, including up to all of the syringe interface devices 14, 214, 314, 414, 514, 614, 714, 814, 914, may be included in a kit, with or without one or more of the syringes 12, that is sold to users. The purchaser of such a kit then has the option of picking which of the syringe interface devices 14, 214, 314, 414, 514, 614, 714, 814, 914 to use with the syringe 12 while administering liquid medication to themselves or to another. If the syringe 12 is reusable, the purchaser of the kid has the option of switching one of the syringe interface devices 14, 214, 314, 414, 514, 614, 714, 814, 914 with another of the syringe interface devices 14, 214, 314, 414, 514, 614, 714, 814, 914 before subsequent uses of the syringe 12.

Although certain illustrative embodiments have been described in detail above, variations and modifications exist within the scope and spirit of this disclosure as described and as defined in the following claims.

1. An adaptive syringe plunger assembly comprising:
a syringe configured to eject fluid, the syringe including a syringe body and a plunger received, at least partially, inside of the syringe body for slidable movement along a plunger axis, the plunger having a first end arranged inside of the syringe body and a second end opposite the first end and arranged outside of the syringe body, and
a plurality of syringe interface devices that are each removably couplable with the second end of the plunger and configured to receive force applied by a syringe user to slide the plunger within the syringe body and eject the fluid from the syringe body,
wherein each of the plurality of syringe interface devices increases a surface area for the syringe user to apply the force to a corresponding one of the plurality of syringe interface devices that is coupled with the second end of the plunger, and each of the plurality of syringe interface devices has a different shape.

2. The adaptive syringe plunger assembly of claim 1, wherein at least one of the plurality of syringe interface devices includes a body and an attachment stem formed to include a hole to receive the second end of the plunger therein, the body includes a top wall and a bottom wall opposite the top wall and the attachment stem extends downwardly away from the bottom wall of the body and is aligned with the plunger axis.

3. The adaptive syringe plunger assembly of claim 2, wherein the body of the at least one of the plurality of syringe interface devices is triangular in shape having three rounded points circumferentially spaced apart from one another relative to the plunger axis and the top wall of the body curves downwardly toward the attachment stem at each of the three rounded points such that an apex of the body is aligned with the plunger axis.

4. The adaptive syringe plunger assembly of claim 2, wherein the body of the at least one of the plurality of syringe interface devices is conical in shape, an apex of the body is aligned with the plunger axis and the top wall extends downwardly away from the apex.

5. The adaptive syringe plunger assembly of claim 2, wherein the body of the at least one of the plurality of syringe interface devices is cylindrical in shape, and the top wall of the body is substantially flat.

6. The adaptive syringe plunger assembly of claim 5, wherein the body includes a side wall extending between and interconnecting the top wall and the bottom wall of the body and the side wall curves radially inwardly toward the plunger axis to form a notch sized to receive fingers of the syringe user therein.

7. The adaptive syringe plunger assembly of claim 2, wherein the bottom wall has a first end and a second end opposite the first end and the top wall extends upwardly away from and is coupled with the first end and the second end of the bottom wall to form a finger-receiving region between the top wall and the bottom wall.

8. The adaptive syringe plunger assembly of claim 2, wherein the body includes a side wall extending between and interconnecting the top wall and the bottom wall of the body and at least a portion of the side wall is formed to include finger-receiving grooves sized to receive fingers of the syringe user therein.

9. The adaptive syringe plunger assembly of claim 2, wherein the hole of the attachment stem of the at least one of the plurality of syringe interface devices is formed to include threads that mate with the second end of the plunger to removably couple the at least one of the plurality of syringe interface devices with the plunger.

10. The adaptive syringe plunger assembly of claim 2, wherein at least a portion of the attachment stem is formed of an elastomer material and the elastomer material is configured to deform to removably couple the at least one of the plurality of syringe interface devices with the plunger.

11. The adaptive syringe plunger assembly of claim 2, wherein the attachment stem is formed to include a first attachment portion and a second attachment portion, the first attachment portion having a tab extending radially away from the first attachment portion and into a tab receiver formed in the second attachment portion to couple the first attachment portion and the second attachment portion together, the first attachment portion and the second attachment portion of the attachment stem cooperate to form the hole that receives the plunger therein and the first attachment portion and the second attachment portion are removably coupled together to removably couple the at least one of the plurality of syringe interface devices with the plunger.

12. The adaptive syringe plunger assembly of claim 1, wherein at least one of the plurality of syringe interface devices includes a hook-and-loop fastener strap formed to include a slit extending therethrough and sized to receive the second end of the plunger therein to removably couple the at least one of the plurality of syringe interface devices with the plunger.

13. The adaptive syringe plunger assembly of claim 1, wherein at least one of the plurality of syringe interface devices includes a body and an attachment stem formed to receive the second end of the plunger therein, the body includes a top wall and a bottom wall opposite the top wall and the attachment stem extends downwardly away from the bottom wall of the body and is aligned with the plunger axis, the bottom wall has a first end and a second end opposite the first end and the top wall extends upwardly away from and is coupled with the first end and the second end of the bottom wall to form a finger-receiving region between the top wall and the bottom wall, and wherein the attachment stem includes a first attachment arm extending downwardly from the body and a second attachment arm radially spaced apart from the first attachment arm and extending downwardly from the body, each of the first attachment arm and the second attachment arm are formed to include a tab extending radially inwardly from a bottom edge of each of the first attachment arm and the second attachment arm and each of the tabs are configured to snap under the second end of the plunger to removably couple the at least one of the plurality of syringe interface devices with the plunger.

14. The adaptive syringe plunger assembly of claim 1, wherein at least one of the plurality of syringe interface devices includes a body, a first attachment arm extending downwardly from the body, and a second attachment arm radially spaced apart from the first attachment arm and extending downwardly from the body, and wherein a tensioning unit extends around the first attachment arm, the second attachment arm, and the plunger to removably couple the plunger with the at least one of the plurality of syringe interface devices.

15. The adaptive syringe plunger assembly of claim 1, wherein at least one of the plurality of syringe interface devices includes a body, a first attachment arm extending downwardly from the body, and a second attachment arm radially spaced apart from the first attachment arm and extending downwardly from the body, and wherein the first attachment arm and the second attachment arm are configured to wrap around the plunger to removably couple the at least one of the plurality of syringe interface devices with the plunger.

16. The adaptive syringe plunger assembly of claim 1, wherein the plunger includes a plunger shaft extending between and interconnecting the first end and the second end of the plunger and having a first diameter, the second end of the plunger is a thumb pad having a second diameter and the second diameter is greater than the first diameter.

17. The adaptive syringe plunger assembly of claim 16, wherein at least one of the plurality of syringe interface devices includes a hollow body and an attachment stem extending downwardly from the hollow body, the attachment stem has a third diameter that is less than the second diameter of the thumb pad and the attachment stem is configured to deform so that the thumb pad passes through the attachment stem and is received in the hollow body.

18. The adaptive syringe plunger assembly of claim 17, wherein the hollow body is triangular in shape having three circumferentially spaced apart points relative to the plunger axis and a top wall of the hollow body curves downwardly at each of the three points such that an apex of the hollow body is aligned with the plunger axis.

19. The adaptive syringe plunger assembly of claim 17, wherein the hollow body is cylindrical in shape and a top wall of the hollow body is substantially flat.

20. The adaptive syringe plunger assembly of claim 17, wherein at least a portion of the attachment stem is formed of an elastomer material and the elastomer material is configured to deform to removably couple the syringe interface device with the plunger.
